(19) United States
(12) Patent Application Publication
(10) Pub. No.: US 2021/0347815 A1
(43) Pub. Date: Nov. 11, 2021

(54) IMPROVED CONTINUOUS EXTRACTION PROCESS FOR THE PRODUCTION OF VEGETABLE PROTEIN CONCENTRATES

(71) Applicant: N.V. Desmet Ballestra Engineering S.A., Zaventem (BE)

(72) Inventors: Marc Kellens, Mechelen-Muizen (BE); David de Schaetzen, Sombreffe (BE)

(21) Appl. No.: 17/286,400

(22) PCT Filed: Oct. 9, 2019
(86) PCT No.: PCT/EP2019/077423

§ 371 Date: Apr. 16, 2021

Related U.S. Application Data
(60) Provisional application No. 62/747,384, filed on Oct. 18, 2018.

(51) Int. Cl. C07K 1/14; A23J 1/14; A23L 11/30; B01D 11/02

(52) U.S. Cl. CPC C07K 1/145; A23J 1/144; A23V 2002/00; B01D 11/023; A23L 11/32

(57) ABSTRACT

This invention relates to a process for the continuous production of vegetal protein concentrates by removing the soluble carbohydrates by an aqueous alcohol from a defatted vegetable material said process requiring no frequent production interruptions for cleaning operations. The innovative process includes the steps of removing at least a fraction of the soluble carbohydrates by solvent extraction, the solvent being aqueous alcohol, to obtain a solvent wet protein rich vegetable material and a miscella; removing the aqueous alcohol form the solvent wet protein rich vegetable material to obtain a vegetable protein concentrate; wherein the solvent extraction takes place in a moving screen solvent extractor equipped with a cleaning means of the moving screen on its return journey and for which no plugging takes place for extended period of time.

### FIELD OF THE INVENTION

This invention relates to a continuous process for the production of vegetal protein concentrates by removing the soluble carbohydrates by an aqueous alcohol from a defatted vegetable material said process requiring no frequent production interruptions for cleaning operations.

### BACKGROUND OF THE INVENTION

Providing abundant and quality food to a continuously growing world population is increasingly challenging. In parallel, the consumption of proteins of animal origin is ever more under pressure notably due to health, ethical, environmental and economic reasons. Therefore, the production of vegetable proteins, such as protein concentrates and to less extend protein isolates, is steadily more attractive.

Because of the relatively high protein content of the defatted meal obtained after the deoiling of some common oleaginous vegetable materials such as soybeans, and to less extend rapeseeds and sunflower, such defatted meal is a suitable starting material for the production of vegetable based, proteinaceous feed and food ingredients such as concentrates and isolates. Currently, soybean protein concentrates are by far the prevalent vegetable proteins available both for human and animal consumption. Protein concentrates will contain typically 60 to 70% of proteins on dry basis (all percentage are expressed weight/weight unless specified otherwise). Thorough removal of soluble carbohydrates from the vegetable material is not only advantageous to produce a protein concentrate of high protein content but is also advantageous for nutritional reasons. Indeed, humans, and many animals do not have the capability to digest some of the complex oligosaccharides contained in the vegetable materials and therefore their removal is advantageous to considerably reduce health issue associated to their incomplete digestion.

Accordingly, a number of processes to produce vegetable protein concentrates have been developed such as for example U.S. Pat. Nos. 3,971,856, 3,268,503, 3,714,210 4,219,470 3,734,901, GB Pat 2 461 093 B. It must be emphasized that the results and tests on which those disclosures are based are sometimes originating from lab scale experimentations. Therefore, their workability in large production facilities is not systematically certain and this is probably why, despite the disclosure of so many processes, currently, soybean protein concentrate is nearly exclusively produced by a process closely derived by the one developed by Hayes many years ago and disclosed in U.S. Pat. No. 3,734,901. In this process, dehulled soybean are first extracted by hexane to remove nearly all the fatty maters. In most instance, the hexane is flash removed from the defatted vegetable material and the resulting desolventized defatted vegetable material that is obtained is further extracted by aqueous ethanol to extract as selectively as possible the soluble carbohydrates. The aqueous ethanol is evaporated from the extracted material and the resulting residue corresponds to soybean protein concentrate having the targeted protein concentration (usually about 60 to 70% of protein, percentage calculated on a dry matter basis). The major variation of the process, as realized in the field, is the technique used for the desolventization of the defatted vegetable material. Indeed, in practice, the protein concentrate is produced in a soybean mill, i.e. a facility equipped with a solvent extractor extracting the oil with hexane and yielding thus, after the removal of the solvent, to crude vegetable oil and defatted vegetable material (also called meal in the industry). If prior to the solvent extraction, the soybeans have been dehulled, the resulting meal has a higher protein content and is an ideal starting material to produce protein concentrate. Such starting material is called “HI-PRO” if the meal desolventization takes place in a conventional desolventizer toaster using a rather large quantity of steam in direct contact with the meal (steam-DT). Due to the combined use of large quantity of steam (and thus moisture) and heat in such conventional DT, the proteins tend to be denatured and have therefore a lower nutritional value. Furthermore, the proteins being denatured, they tend to be more soluble and hence some of them can be lost in the solvent extraction with aqueous alcohol which lead to protein concentrate with lower protein content. The starting material is called “WHITE-FLAKES” if the desolventization of the defatted material takes place in a vacuum desolventizer (vacuum-DT) with minimum steam injection in direct contact with the meal. In those conditions, the obtained material is white, fluffy, contains more dust and fines than the HI-PRO but their proteins are less denatured because of the limited contact with the steam (water) at high temperature. WHITE-FLAKES is the starting material of choice for high quality protein concentrates, in particular protein concentrates suitable for human consumption. However, the process according to the present invention is advantageous for those two starting materials. Indeed, both materials (HI-PRO meal or WHITE-FLAKES) will lead to percolation problems that are fully solved by the process according to the present invention: white-flakes is a fluffy material containing a lot of fines, dust and small particles having thus a tendency to plug the extractor and reduce the percolation; HI-PRO is a coarser material containing less fines but as explained above, some of the proteins will be extracted by the aqueous alcohol and those proteins are sticky and have also a marked tendency to plug the extractor and reduce the percolation rate.

The oil extraction can however also be performed by mechanical pressing. Mechanical pressing is more expensive but may be perceived as a greener process by the public and has the additional advantage of not requiring a solvent evaporation step.

The process currently in use commercially, as well as all cited processes have in common they contain at least one extraction step involving the extraction of soluble carbohydrates with an aqueous alcohol. This extraction of soluble carbohydrates with aqueous alcohol will be mandatory for other oleaginous vegetable material as well, such as rapeseed, sunflower but also for non-oleaginous vegetable material such as navy bean (Phaseolus vulgaris), lentil (Lens esculenta), broad or faba bean (Vicia faba) because all those vegetal materials contain substantial amount of soluble carbohydrates.

However, it is observed that the production of vegetable protein concentrates is difficult due to low percolation rate even if the most commonly used extractor, i.e. a shallow bed fixed screen loop extractor is used. Indeed, intense manual cleaning of this extractor, in particular its fixed screen, must be done regularly, typically one to two times per month. This is due to the progressive clogging of the fixed screen. It has been reported in the field that the frequency of such aggressive manual cleaning can be about twice per month. Such frequent regular intensive cleaning operations are not only costly due their considerable impact on the productivity but also raise most serious safety issues principally because those manual cleaning involve the manipulation of flammable solvent able to potentially cause fatal explosions. Therefore, it would be highly advantageous to design a process requiring no frequent manual cleaning operation of the screen.

In an attempt to remedy to this progressive plugging of the fixed screen, WO/2015/179530 to Floan et al. discloses an “Extractor with screen washing system”. This disclosure describes an extractor comprising: an extraction chamber; a conveyance system to convey solid materials through the extraction chamber in a direction of material travel; a screen to support the solid materials as they are conveyed by the conveyance system, the screen supported by a screen frame; a fluid supply system disposed above the solid materials and configured to apply a fluid to the solid materials; a fluid removal system disposed below the solid materials and configured for removing the fluid after it has passed through the solid materials and the screen; and a screen washing system disposed under the screen and supported against movement in the direction of material travel, the screen washing system including a washing fluid intake and a plurality of outlet nozzles directed upward towards the screen.

This development contains thus a screen, more precisely a fixed screen and thus, when the extractor is in operation, only the bottom side of the screen can be cleaned by the disclosed screen washing system, in particular a plurality of outlet nozzles directed upwards towards the screen. Furthermore, in order to continuously clean the bottom side of the fixed screen, either the washing system must extend over the total surface of the screen(s) which lead to large solvent consumption or a single washing system must move over the whole length and width to reach all the bottom surface of the screen(s) leading to mechanical complexity. Another disadvantage is that the cleaning fluid mixes with the miscella and dilute it which impose strict limitation to its choice and to its output. Furthermore, the industrial experience has shown that even if the washing system disclosed in WO/2015/179530 induces a significant reduction of the plugging of the fixed screen(s), a manual cleaning of said fixed screen(s) must still be done frequently, thus still impacting very negatively the attractivity of the process.

It is believed that the practical production difficulties described here above are in part responsible for the still relatively small volume of soybean protein concentrate (SPC) annually produced compared to the huge volume of available starting material. Indeed, the worldwide annual production of soybean is estimated at 320 Million tons in 2017, corresponding to about 220 Million tons of meal suitable as starting material for the production of SPC. However, it is estimated that only about 3.5 Million tons of SPC has been produced in 2017. This relatively modest volume, compared to the volume of meat produced on a worldwide basis (330 Million tons in 2017), is certainly not due to a lack of available starting material, but rather to a lack of an available robust SPC production process.

Therefore, despite the merits of the prior art, a need exists for a robust process for the production of vegetable protein concentrate, in particular for a process requiring no frequent manual and aggressive cleaning necessitating recurrent interruptions of the production process.

### Object of the Invention

It is an object of the invention to provide a process for the production of vegetable protein concentrate from a defatted vegetable material said process being robust, in particular said robust process requiring no manual cleaning in order to reestablish proper aqueous alcohol percolation during an extended period of time, i.e. during a period of continuous operation of at least two months.

A further object of the invention is to provide a process for the production of vegetable protein concentrate from a defatted vegetable material said process involving no mixing of the cleaning solvent(s) with the miscella.

### SUMMARY OF THE INVENTION

It has been surprisingly found that a robust process, for the production of vegetable protein concentrate from a defatted vegetable material including the steps of firstly removing at least a fraction of the soluble carbohydrates of said defatted vegetable material by solvent extraction, the solvent being aqueous alcohol, to obtain solvent wet protein rich vegetable material and a miscella and secondly removing the aqueous alcohol solvent form the solvent wet protein rich vegetable material to obtain a vegetable protein concentrate, is achievable when the solvent extraction takes place in a moving screen extractor equipped with a specially designed cleaning means of said moving screen during its return journey said cleaning means including contacting said moving screen with at least one cleaning solvent and said cleaning means insuring a complete cleaning of both sides of said moving screen during the return journey of said moving screen and thus avoiding the progressive plugging of said moving screen therefore insuring continuous operation of the moving screen extractor for an extended period of time.

In another aspect of the invention, the process as previously described is further characterized by a extended period of continuous operation of the moving screen extractor superior to two months.

In another aspect of the invention, the process as previously described is further characterized by a cleaning means including the use of sprayers projecting cleaning solvent towards said moving screen.

In another aspect of the invention, the process as previously described is further characterized by a cleaning means being installed at the very beginning of the return journey of said moving screen.

In another aspect of the invention, the process as previously described is further characterized by repeating the cleaning of said moving screen two or more times by installing two or more cleaning means in series on the return journey of said moving screen.

In another aspect of the invention, the process as previously described is further characterized by utilizing a cleaning solvent having the same composition than the aqueous alcohol used as extraction solvent for the soluble carbohydrates contained in the defatted vegetable material.

The process according to claim 3 wherein said cleaning solvent has the same composition than the aqueous alcohol used as extraction solvent for the soluble carbohydrates and is recycled in the last extraction zone of said solvent extractor.

In another aspect of the invention, the process as previously described is further characterized by the nature of the cleaning solvent being water.

In another aspect of the invention, the moving screen cleaning means can be a combination of both solvent spraying and a mechanical device such as brush and/or scrapper.

In another aspect of the invention, the process as previously described is further characterized by the pressure of the solvent spray being pulsating, alternating for example from very low pressure comprised between 0.1 to 5.0 bars to higher pressure comprised between 5.0 and 100.0 bars.

In another aspect of the invention, the process as previously described is further characterized by using a cleaning solvent bath and immerging at least a part of the moving screen in the cleaning solvent bath said immersion occurring during the return journey of said moving screen.

In another aspect of the invention, the process as previously described is further characterized by the vegetable defatted material being obtained from the deoiling of oleaginous vegetable material such as soybeans, sunflower or rapeseed.

In another aspect of the invention, the process as previously described is further characterized by the vegetable defatted material being selected amongst legumes such as lentils, beans or peas said legumes having optionally been pretreated with treatment such as dehulling and/or flaking and/or cooking.

In another aspect of the invention, the process as previously described is further characterized by the moving screen being cleaned and maintaining its nominal solvent percolation capacity by sprays of cleaning solvent projected on said moving screen during its return journey and wherein the cleaning solvent does not mix with said miscella.

In another aspect of the invention, the process as previously described is further characterized by the moving screen being simultaneously cleaned on both side of the screen; the top surface of the moving screen (on which the extracted material was loaded) and the bottom part of the moving screen (on which no material is loaded).

In another aspect of the invention, the process as previously described is further characterized by the moving screen being cleaned and maintaining its nominal solvent percolation capacity by upwards and downwards sprays of cleaning solvent projected on said moving screen during its return journey.

In another aspect of the invention, the process as previously described is further characterized by the moving screen being cleaned and maintaining its nominal solvent percolation capacity by downwards sprays of cleaning solvent projected on said moving screen during its return journey.

In another aspect of the invention, the process as previously described is further characterized by a cleaning means including at least one rail of cleaning solvent sprayers, said rail being essentially perpendicular to the movement of said moving screen.

In another aspect of the invention, the process as previously described is further characterized by a cleaning means including a plurality of cleaning solvent sprayers, said sprayers projecting cleaning solvent at a pressure ranging from 0.1 to 100 bars.

In another aspect of the invention, the process as previously described is further characterized by a cleaning means including a plurality of cleaning solvent sprayers, said sprayers projecting cleaning solvent at an output of 150 to 1500 liters per minutes and per M².

In another aspect of the invention, the process as previously described is further characterized by a mechanical cleaning means including scrapers and/or brushes in combination with sprayers.

In another aspect of the invention, the process as previously described is further characterized by the presence of an expending zone located right after the feeding inlet of the extractor.

In another aspect of the invention, the process as previously described is further characterized by introducing the starting material on the moving screen of the moving screen extractor in a way to cover only a fraction of the width of said moving screen, said starting material being subsequently wetted by the extraction solvent and allowed to swell freely at least in the transversal direction. The starting material introduced on the moving screen of the moving screen extractor can be dry or already wetted by a solvent such as concentrated alcohol, aqueous alcohol or hexane for example.

### BRIEF DESCRIPTION OF THE DRAWINGS

FIG. 1 shows a longitudinal view of a shallow bed fixed screen solvent loop extractor. The extraction takes place on two fixed screens.

FIG. 2.a shows a longitudinal view of a moving screen extractor suitable for the process according to the present invention. The extraction takes place only on the top part of the moving screen only. The extractor is equipped with cleaning means of the moving screen during its return journey.

FIG. 2.b shows a transversal view of the moving screen extractor to illustrate how the miscellas are deviated on the sides and do not contact the moving screen during its return journey.

FIG. 3 shows a top view of a moving screen extractor suitable for the process according to the present invention and illustrate an optional procedure to introduce the starting material into said extractor.

### DEFINITIONS

The term “miscella”, as used in disclosing the present invention, refers to a solution of oil or soluble carbohydrates in solvent(s) such as resulting from a solvent extraction process.

The terms “vegetable protein concentrate”, as used in disclosing the present invention, encompass a derivative of a defatted vegetable material with an increased protein content obtained after the extraction of at least a fraction of the soluble carbohydrates from said defatted vegetable material by an aqueous alcohol solution in a percolation extractor. Typical protein content of vegetable protein concentrate is 60-70% (on a dry basis) but depending on the precise vegetable material and target application this percentage can slightly differs from this average range.

The terms “soluble carbohydrates”, as used in disclosing the present invention, encompass a broad range of compounds soluble in aqueous alcohol including for example simple carbohydrates such as fructose and glucose or complex carbohydrates or oligosaccharides such as saccharose, raffinose, stachyose or polysaccharides such as starch. The exact composition will depend on the processed vegetable material. For example, in general, soluble carbohydrates will be rich in saccharose, raffinose, stachyose for oleaginous material and rich in starch for legumes. Again, depending of the precise type of processed defatted vegetable material and the target protein content of the final protein concentrate, the quantity and the nature of the extracted soluble carbohydrates will vary considerably.

The terms “vegetable defatted material”, as used in disclosing the present invention, encompass the defatted meal obtained after the deoiling of oleaginous vegetable material such as but not limited to soybean, rapeseed and sunflower. The deoiling can be mechanical pressing or solvent extraction or realized by combination of both. The solvent is often hexane, but other solvents may prove useful as well. Other solvent may be for example concentrated alcohol (dry alcohol) or methyl-tetrahydrofuran. The terms “vegetable defatted material” also encompass legumes such as lentils, beans and peas. Those legumes may have undergone one or more pretreatments such as dehulling, grinding, classification, flaking and/or cooking or any combinations of those treatments. Typically, a defatted vegetable material contains less than 2% of fatty matters. However, this value is only indicative, and our invention is not limited by this typical value of fatty matters present in the defatted vegetable material.

The terms “starting material” include any vegetable defatted material either in a dry form or already wetted by any solvent such as the extraction solvent that can be used for the extraction of carbohydrates (for example aqueous alcohol) or such as the extraction solvent that can be used for the extraction of oil (for example hexane or concentrated alcohol).

The terms “moving screen extractor” refer to solvent extractor designed originally to perform the hexane extraction of material having inherently a fairly good solvent percolation. Such moving screen extractor is for example used for the hexane extraction of the oil contained in various oil seeds such as soybean, rapeseed and sunflower. A typical moving screen extractor useful for the process according to our invention is the LM (standing for Low Maintenance) and originally developed by “Extraction De Smet” of Belgium (now Desmet Ballestra of Belgium). It must be stressed that such moving screen extractor is of extremely sturdy construction including the moving screen which is preferably constructed in high grade stainless steel such as the SS304 grade for example. Such moving screen is so sturdy it can operate several decades without overall. In the field, those extractors are often designated by “LM Extractors” even if they were manufactured by companies other than Desmet Ballestra.

The terms “fixed screen extractor” refer to an extractor where the screen is fixed and the material to be extracted is pushed forward by vertical paddles or pushers. This principle allows the loop construction including thus two stacked fixed screens, such configuration allowing thus a large extraction area for a small foot-print. This type of extractor (using two stacked fixed screen) is particularly suitable for process requiring limited (low) bed height. But fixed screen extractor exists also in one stacked fixed screen with deep bed height. However, the present invention is not applicable to any fixed screen extractors.

The terms “robust process” refer to a process that can be continuously operated for extended period of time typically longer that 2 months such as for example 3 months, 4 months, 5 months, 6 months or more. In particular, a robust process does not require manual cleaning of the fixed screen during its continuous operation period.

### DETAILED DESCRIPTION OF THE INVENTION

The current production of vegetable protein concentrates, such as for example soybean proteins concentrate, make use of two extractions, a first extraction of oil (in most of the case a solvent extraction with hexane) followed by the extraction of most of the soluble hydrocarbons with aqueous alcohol. In this process, the second extraction, with aqueous alcohol is relatively difficult because of poor percolation of the aqueous alcohol. This poor percolation is due to the fact that the material extracted is quite compacts and is made of small particles containing a lot of fines. To make the situation worse, fines contains a large proportion of proteins and when in contact with the water contained in the aqueous alcohol used for the extraction, a fraction of those fines will form a viscous glue. Therefore, the viscous glue and the remaining fines, dust, small particles start to accumulate on critical parts of the solvent extractor, i.e. on the fixed screen of the extractor and furthermore decrease the solvent percolation. Furthermore, this material accumulating on the fixed screen of the extractor, with the course of time dries and becomes a very hard material extremely sticking to said fixed screen. It can only be removed by highly intense cleaning technique such as mechanical scrapping and/or high-pressure spray of 150 bars for example. The extracted soluble carbohydrates are also sticky which will not help the situation. Furthermore, a small part of the proteins is solubilized by the aqueous alcohol. Those solubilized proteins are also known to be sticky. Consequently, the percolation decreases progressively due to the plugging of the fixed screen. After some time of operation, as short as two weeks in some instances, the percolation rate of the aqueous alcohol has decreased so much that a manual cleaning of the plugged part(s) of the extractor is necessary. Of course, such manual cleaning requires to stop the production. This is in sharp contrast with most if not all of the other processes (such as for example oil extraction, refining) in use for the processing of vegetable oleaginous material which are running 24 hours a day for extended period of time such as one year for example. It is believed that this major difficulty is a strong deterrent for companies to invest in vegetable protein concentrates production lines.

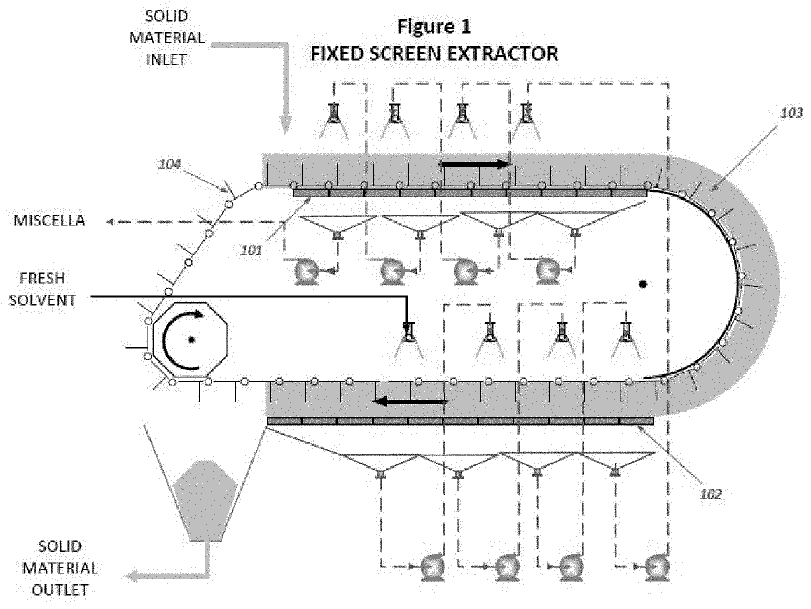

When a material is known to have such extremely poor solvent percolation, the solvent extractor of choice is a shallow bed fixed screen loop extractor, as represented on FIG. 1. Such solvent extractors are specifically designed to solvent extract low layer of material and are known to be less sensitive for material having extremely poor percolation. Furthermore, the fixed screens (101 and 102) have the apparent advantage of being constantly rubbed by the extracted material (103) that is constantly pushed forward by the moving pushers or paddles (104). This rubbing effect has of course a mechanical cleaning action on the bars or grids forming the fixed screens (101 and 102) at least for the side in direct contact with the processed material. In the field this rubbing effect is considered to be advantageous concerning the plugging compared to moving screen extractor. As a matter of fact, they are considered to be self-cleaning. Another advantage of the loop configuration of the extractor currently used is that it makes possible the extraction on two fixed screens, a first one (101) making the top part of the loop and a second one (102) making the bottom part of the loop. The surface exposed to the solvent extraction is therefore optimized in regard to the size of the apparatus is particular its footprint.

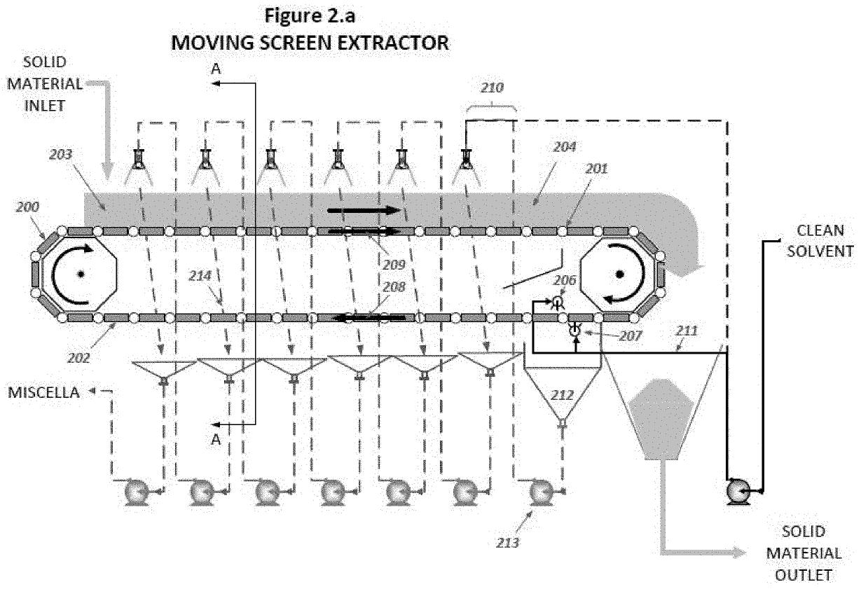

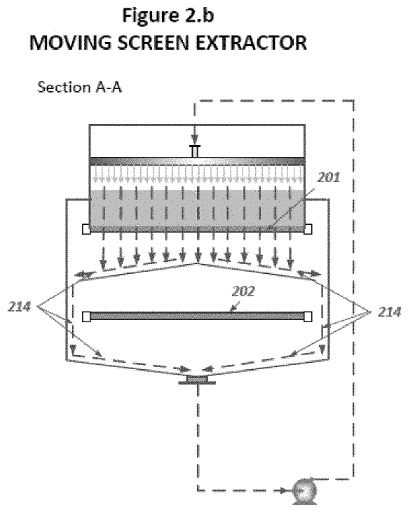

Another type of percolation solvent extractor used in the oleaginous industry is the moving screen extractor as represented on FIGS. 2.a and 2.b. However, a moving screen extractor is normally used with material having inherently a sufficient percolation and for which the percolation will remain sufficient even for deep bed of material. Sufficient percolation means is this context that the solvent is able to percolate down the material in a limited amount of time so that productivity of the extractor can be attained. Furthermore, by conception a moving screen extractor can only carry out the extraction on one side of the moving screen (200) i.e. the top deck (201). In the top deck (201) part of the extractor, the moving screen moves (209) from the solid material inlet to solid material outlet (211). The actual extraction is thus performed in the top deck. In the bottom deck (202) of the moving screen simply moves back (208) from solid material outlet to the solid material inlet (203) of the extractor and has no extraction functionality during this return journey. Therefore, for a given thickness of an extracted layer, a moving screen extractor will have only about 50% of the capacity of a fixed screen loop extractor having a similar foot-print. Furthermore, in a moving screen extractor, the material that is extracted (204) simply sits on top deck part of the moving screen and has thus no mechanical rubbing effect. This situation is totally recognized in the field to the extent that most producers of soybean protein concentrates favor fixed screen shallow bed loop extractors for the extraction of soluble carbohydrates. Indeed, to our knowledge, the current manufacturers of soybean protein concentrates utilize mostly shallow bed fixed screen loop extractors for the extraction of soluble carbohydrates with aqueous alcohol. The few other manufacturers that are not using such type of extractor may use solvent batch extractors or other types of extractors.

It must be stressed that in the case of the moving screen extractor, the miscellas are collected directly below the top deck. Thus, the miscellas do not percolate through the bottom deck and thus is not in contact with the moving screen during its return journey. On the FIGS. 2.a, the dashed arrows 214 are oblique to indicate that the miscellas are deviated on the sides of the extractor to be collected and recirculated adequately, i.e. in the next extraction zone to perform an extraction in a counter-current mode. Indeed, those collecting and recirculating pieces of equipment are bulky and technically difficult to fit between the two decks. FIG. 2.b shows a transversal view of the same moving screen extractor along a section A-A and illustrates how the miscellas (214) dripping from the top-deck (201) are deviated on the sides of the extractor, collected and recirculated in the next extraction zone to perform an extraction in a counter-current mode. The bottom deck (202) of the extractor is therefore not in contact with the miscellas which also contribute to keep it clean. The deviation of the miscellas is usually performed by a Chinese-hat metallic structure.

It has been surprisingly found that no manual cleaning of the extractor is needed for extended period of time if the aqueous alcohol extraction step is performed in a moving screen extractor equipped with at least one cleaning device, preferably a continuous cleaning device even if theoretically this type of moving screen extractor is expected to give the poorer extraction performance and very frequent manual cleaning since they cumulate two defects:

1) The material extracted simply lays on the moving screen when being extracted by the aqueous alcohol. Hence, there is no scrubbing effect of this material on the moving screen as it is the case for a fixed bed extractor (also known in the field as self-cleaning extractor). In this situation it is expected to face a very rapid plugging of the moving screen and unacceptable frequent reduced percolation asking for a manual cleaning of the equipment.

2) They are of the non-loop type meaning that actual extraction is performed on a surface roughly half the one available on loop extractors meaning that for a given output and for a given (low) layer of material bed height, a moving screen extractor should have a footprint two times larger which is more expensive. Up to now, the vegetable proteins producers use low layer material to cope with the poor percolation of the material and risk of further reduced solvent percolation due to the progressive plugging of the screen. This risk is further considered in case of no scrubbing effect as mentioned above.

However, is has surprisingly been observed that no plugging of the moving screen takes place even after two months or more of continuous operation when the moving screen is cleaned with a solvent such as aqueous alcohol or water, said cleaning typically involving rails of solvent sprayers (206 and 207) installed perpendicularly to the moving screen and installed on the return journey (208) of the moving screen. Preferably both sides of the moving screen are contacted continuously with an adequate cleaning solvent. The cleaning solvent can for example be the aqueous alcohol used to extract the soluble carbohydrates from the defatted vegetable material. Instead of providing the aqueous alcohol directly on top of the defatted vegetable material in the last extraction compartment (210), the aqueous alcohol or at least a part of it (211) serves the purpose of cleaning the moving screen during its return journey. Said aqueous alcohol, is of course integrally recovered (212) and pumped (213), along with the residue, on top of the defatted vegetable material in the last extraction compartment (210). This embodiment has the supplementary advantage that the overall solvent consumption does not increase and does not modify the required optimum of alcohol to water ratio during the complete process of extraction and furthermore does not require additional water consumption for a process that is already water demanding. If the cleaning solvent is water, the water once its cleaning action performed is recovered and not mixed with the miscella. Optionally the water is treated for example by decantation and/or filtration and at least part of this treated water is recycled as cleaning solvent. This has the advantage to reduce the amount of water to be added in such water demanding process. It must be stressed that, compared to the cleaning of a fixed bed, the miscella is not diluted by the cleaning solvent which is highly advantageous since it offers much more flexibility for the choice of said cleaning solvent and the modalities of its use such as its output and its temperature for example. Compared to the cleaning of a fixed screen which can be cleaned by solvent spray only on the bottom side because the other side, i.e. the upper surface, is constantly loaded with vegetable material, the cleaning of the moving screen occurs during its return journey, i.e. during a phase for which the moving screen is not loaded with vegetable material. Hence the cleaning can take place on both sides of said moving screen and the output of the cleaning solvent can be set freely so that it removes any traces of fresh proteins, fines, carbohydrates or mixture thereof. Consequently, the moving screen, after the cleaning, is perfectly clean and keeps its nominal solvent percolation.

Preferably the moving screen is continuously cleaned and maintains its nominal extraction solvent percolation capacity by upwards and downwards sprays of cleaning solvent projected on said moving screen during its return journey, optionally said solvent cleaning can be assisted by mechanical cleaning devices like scrapers and/or brushes. However, good cleaning results have been observed by exclusive downwards sprays of solvent projected on said moving screen, more precisely on the top-side of the moving screen during its return journey. It has been observed that the output of the cleaning solvent must be sufficient to detach and entrain all residue. Cleaning solvent is typically 750 liters per minute and per M². However, it is expected that this optimal cleaning solvent output can vary greatly depending on many parameters such as the exact processed vegetable material but also the design and the material of the moving screen.

It has also been observed that if the cleaning solvent spraying on the moving must be interrupted for some reasons for a short period (a few minutes for example), optimal percolation could be resumed by starting again the cleaning of the moving screen. However, if the cleaning solvent sprayings on the moving is interrupted for a longer period (in excess of 6 hours for example), it may be somewhat more difficult to regain an optimal percolation by simply turning on the cleaning of the moving screen with sprays of cleaning solvent. Combined use of cleaning solvent sprayings and mechanical device(s) may be needed for those instances.

In the context of the present invention, the pressure of the spray of the cleaning solvent can range for example from 0.1 bar to 5 bars. However, our invention is not limited to this pressure range. Sufficient cleaning can occur with pressures lower than 0.1 bars. For example, it has been observed that the moving screen can be perfectly cleaned by a simple immersion of at least a portion of the moving screen in a cleaning solvent bath. On the other hand, depending on the starting raw material and various other conditions, in some circumstances, a cleaning solvent spray having a pressure substantially higher than 5 bars may be required, notably if the cleaning has been interrupted for any reasons for some time and that a subsequent build-up of residue had the time to accumulate and started to solidify.

In another embodiment of the process according to our invention, the cleaning of the moving screen occurs at the very beginning of its return journey of the moving screen. Indeed, it has been observed that the sooner the cleaning was realized, the easier any residue was removed by the cleaning solvent.

In another embodiment of the process according to our invention, the cleaning of the moving screen is repeated two or more times by installing two or more cleaning means in series similar to the ones already depicted (206, 207, 211, 212, 213) on the return journey of the moving screen.

In another embodiment of the process according to our invention, the pressure of the solvent spray is pulsating alternating for example very low pressure and higher pressure.

In another embodiment of the process according to our invention, the direction of the solvent spray is oscillating.

In another embodiment of the process according to our invention, only the top side of the moving screen is cleaned by sprays of cleaning solvent.

In another embodiment of the process according to our invention, the starting material is introduced in a dry form into the moving screen extractor and cover only a fraction of the width of the moving screen, for example 60 to 70% of the width. The starting material in then contacted by extraction solvent and allowed to swell in to swell a least in the transversal direction.

In another aspect of the invention, the process as previously described is further characterized by using a cleaning solvent bath and immerging at least a part of the moving screen in the cleaning solvent bath said immersion occurring during the return journey of said moving screen.

Starting Material

The starting material of the process according to the present invention is defatted vegetable material and encompasses a large variety of materials. The defatted vegetable material can notably result from the oil extraction of a large variety of oleaginous vegetable material. The oil extraction can involve mechanical or solvent processes or their combination. Preferably, prior to the oil extraction, the vegetable oleaginous material has been adequately prepared and dehulled to remove a substantial fraction of insoluble fibers. The defatted vegetable material can also belong to the legume family. Oil content of legumes are naturally low, usually below 2% and therefore those vegetable materials are naturally defatted. However, legumes may have undergone one or more pretreatment such as dehulling to remove a substantial fraction of insoluble fiber, flaking, grinding, cooking or the combination of some of those treatments. Those pretreatments are known to generate fines, dust that are detrimental for solvent percolation. However, the process according to the present invention is able to cope with fines, dust and provide a solution to reduce the inconvenient induced by said fines and dust. Each treatment increases the amounts of dust, fines and other small particles. It must be understood that dust, fines and other small particles are present in rather large quantity in defatted vegetable materials and constitute a real major problem for the solvent percolation hence the very feasibility of the extraction. This major problem is even amplified when the extraction of soluble carbohydrates with aqueous alcohol is undertaken because the solubilized carbohydrates are sticky on their own. Furthermore, a small quantity of proteins may be solubilized as well, and proteins are also known to be sticky, in particular is presence of water they will form a very thick glue along with the fines which in fact contain predominantly proteins. In conclusion, the process according to the present invention surprisingly provides an efficient solution to a major issue faced since many years by the industry.

The starting material is introduced by the solid material inlet of the moving screen extractor by being dropped, by gravity, on the moving screen. The starting material can be introduced in a dry form or already wetted by a variety of solvent. It must be specified that in the context of this invention and thus in the context of vegetable material, dry as the meaning of sufficiently dry to be stored for extended periods of time without degradation. Thus, dry means that the moisture content of the material is below a limit, 10% for example. This moisture content limit will be variable in function of the type of vegetable material.

Usually the starting material is introduced already wetted by the extraction solvent (aqueous alcohol for example). However, the starting material that is introduced in the moving screen extractor could have been wetted by other solvents, for example concentrated alcohol. Concentrated alcohol can be used to extract the oil contained in any vegetable material. The advantage of using concentrated alcohol, even if the solvent in more expensive than hexane, for the extraction of oil, is that not solvent evaporation is needed since the solvent wet material can be introduced straight into the moving screen extractor where the carbohydrate will be extracted. Indeed, as aqueous alcohol is used for this extraction, the concentrated alcohol is simply displaced by the extraction solvent.

However, even if the starting material is wetted by any solvent it will still be solid and behave as a solid material. Attention must be provided to its handling to maintain proper solvent percolation in the downstream steps of the process according to the present invention.

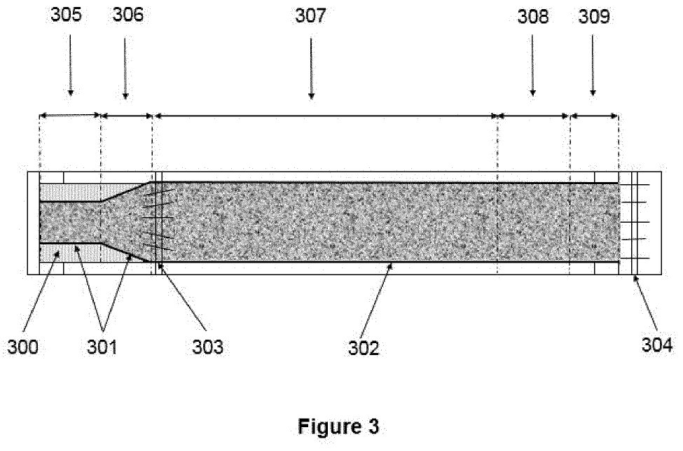

It has been observed that the procedure used to introduce the starting material in the solid material inlet of the moving screen extractor is highly relevant to the solvent percolation rate. It has surprisingly been observed that if the solid material is introduced so as to cover only about 60 to 70% of the width of the moving screen, the percolation rate, and thus the output of the extractor can be substantially increased. This counter intuitive observation may be due to the marked swelling of the starting material when contacted with aqueous ethanol used as extraction solvent. Indeed, without willing to be bound by any theory, it is believed that if the starting material occupies the full width of the moving screen before being contacted by the aqueous ethanol, the material will not have the possibility to expand laterally when contacted by said aqueous ethanol and will be therefore compacted and even less permeable to solvent. The lateral expansion is of course limited by the metallic sides of the extractors which match to the width of the moving screen. Means to introduce the solid material on the moving screen so as it covers only about 60 to 70% of its width can simply consist of vertical metallic plates delimiting the allowed and wished loading section width of the moving screen. Said vertical plates are for example just below the solid material inlet, from this location said vertical plates are progressively widening to reach the final width of the moving screen. With such means, the starting material has to possibility to swell and expend naturally laterally when contacted with the aqueous alcohol. This procedure to introduce the starting material into the moving screen extractor is particularly beneficial for the production of vegetable protein concentrates because it may increase a usually low solvent percolation rate. This observation has been made during the production of soybean protein concentrate but it is expected that a similar behavior will occur for alternative vegetable material. Indeed, the swelling is a normal behavior of proteins contacted with water. However, its amplitude may depend on the nature of the starting material and therefore its actual introduction procedure may be adapted. In particular the percentage of the moving screen covered by the starting material may be adapted and therefore the vertical metallic plates delimiting the allowed and wished loading section width are preferably adjustable from the outside of the extractor. FIG. 3 represents a top view of the moving belt extractor that may be used in the process according to the present invention and to this embodiment. In FIG. 3, the unloaded area of the top-deck (300) is delimited by adjustable vertical walls (301). Preferably, those adjustable walls are adjustable from outside. Those adjustable walls widen to reach the fixed walls (302) of the extractor. Optionally a meal layer spreader (303) is used to even out the thickness of the meal layer loaded on the top-deck of the moving screen extractor. Therefore, according to this optional feature, the moving screen extractor has a narrowed solid material feed inlet (305) than the full width of the moving screen, a wetting and swelling section (306), an extraction section (307), optionally a displacement section with concentrated alcohol (308) and a solvent drainage section (309). At the end of the top-deck, the solvent-wet extracted material drops to but further processed.

It must be stressed that this starting material introduction procedure is much more difficult to apply in the case of a fixed screen extractor. Indeed, as seen on FIG. 1, the pushers (104) are in the way and no vertical plates can be fitted close to the fixed screen per se.

Example of Starting Material

The following is a non-limiting example that will precisely illustrate, in the case of soybeans, the extend of the various pretreatments, each of them generating its share of fines, dust and small particles, that will result in defatted vegetable material suitable for the process according to the present invention. The choice of soybean as an example is made in accordance with its prevalence in the market. The typical composition of soybean on a dry basis is around 40% protein, 30% carbohydrates and phosphatides, 20% triglyceride oil and 10% ash and fibers. Raising the protein content therefore entails the removal of other constituents. If the hulls and oil are removed by dehulling and extraction, the protein content in the resulting meal containing 13% moisture, is increased to 45-48%. Producing protein concentrate by removing soluble carbohydrates (oligosaccharides such as saccharose, raffinose and stachyose) from the defatted and dehulled meal by extracting the meal with aqueous ethanol can raise its protein content further, generally to some 60-70% on a dry basis.

Thus, the harvested soybeans are first dehulled and preferably the dehulled beans undergo a pre-treatment to rupture their cell walls. This pre-treatment may comprise for example a cracking and a flaking of the beans. In this treatment, the oilseeds are compressed between two or more cylindrical cracking rollers, dehulled and then further and flattened between two large flaking rolls into flakes. Before this treatment, the dehulled beans may have been conditioned by a heat treatment to soften the material and facilitate the release of the hulls from the meat and thus reduce the energy requirement of the actual flaking process. Because of the compression and shearing forces involved in this flaking process, almost all cells containing the oil are opened by rupture of their walls. This opening of the cells greatly facilitates extracting the cell contents by avoiding the need for the solvent to diffuse through cell walls. A typical flake thickness aimed for in the case of soybean is 0.3 to 0.5 mm according to the type of selected solvent extractor.

Another pre-treatment causing cell walls of the soybean to be ruptured is screw pressing since this also entails strong compression and shearing forces. Screw pressing does not remove all oil from the oleaginous vegetable material so the resulting press cake still contains a substantial amount of oil and this one is recovered by a hexane extraction. It can be extracted as such or after having been pelletized since this treatment also causes further cell walls to be ruptured. In addition, the pellets have better percolation characteristics than the original press cake. Alternatively, an expander maybe used to pre-treat the dehulled soybeans.

The next step leading to one of the starting material suitable for the process according to the invention, is the oil extraction of the pre-treated material, typically a solvent extraction with an apolar solvent such as hexane, which is the name given to an industrial petroleum fraction consisting primarily of C6 saturated hydrocarbons such as n-hexane, methylpentanes, methylcyclopentane, etc. Such extraction of the lipids can be carried out in various solvent extractors, for example a rotating fixed bed extractor. The typical operating temperature of this step will be just below the atmospheric boiling point of hexane (62° C.). Accordingly, an oil-miscella strength of for instance 25-30% of oil in the solvent and a solvent-wet defatted vegetable material containing for example 25-30% of solvent are obtained. It is common that the residual oil content of the defatted vegetable material is less than 1% by weight or even less than 0.5% by weight. Of course, this defatted material is solvent wet and must be desolventized either is a steam DT or a vacuum DT after a preliminary solvent flash, as described in detail before. It is common that, after all those treatments, the fines, dust and other small particles of these defatted materials amounts to 10 to 20%.

Example of Alternative Raw Materials

However, sunflowers and rapeseed, in particular the defatted meals obtained after their oil extraction, are also attractive starting materials for the production of protein concentrates. The techniques to be used are similar even if adaptations are to be expected of course. In particular more soluble carbohydrate must be extracted because sunflower and rapeseed contain less protein than soybean. Moreover, the market acceptance is a challenge for the success of protein concentrates form rapeseeds, sunflowers and/or other vegetable materials. Furthermore, in order to increase the concentration of protein, it is mandatory to remove, prior to the oil extraction, the hulls of those seeds, and currently this can be done consistently for sunflower and soybeans. It is applicable in case of rapeseed, but the oil content in the hulls fraction remain too high, therefore, an additional process for the oil extraction of the hulls fraction is needed which increase the capital investment and the complexity.

More recently, other vegetable materials that are not oleaginous have been proposed as starting material for the production of protein concentrates, in particular legumes such as navy bean (Phaseolus vulgaris), lentil (Lens esculenta), broad or faba bean (Vicia faba). Protein concentrates can be obtained by specific grinding and classification of those legumes, but the yield and the protein content of the concentrates obtained by currently available mechanical techniques remain low. Therefore, the process according to the present invention is also advantageous for legumes and will provide concentrates having the market-expected high protein content of 60 to 70% for example, with high yield.

As a matter of fact, the process according to the present invention will be advantageous regardless the starting material as long as said starting material necessitates an aqueous alcohol extraction step to remove a substantial fraction of the soluble carbohydrates and hence increase the residual protein concentration of the residue.

Cleaning Solvent

The cleaning solvent can be the solvent used for the extraction of the soluble carbohydrate from the defatted material. It permits its recycling as extraction solvent. However, the fact that this cleaning solvent is not mixed with the miscella, result in more flexibility for its choice. For example, water can be used as the cleaning solvent. It that case, the residual water can be discarded, filtered and recycled as cleaning solvent or at least partially used to dilute the alcohol at the desired concentration. The temperature of the cleaning solvent is usually the temperature of the aqueous alcohol used for the extraction of soluble carbohydrates, for example 70° C. However, in some circumstance, temperature can be lower, for example water of about 20-25° C. which is the usual tap water temperature. It must be stressed that the cleaning of a fixed bed offers much less flexibility concerning the cleaning solvent choice because said cleaning solvent will be automatically mixed with the miscella and dilute it.

It is also possible to clean the moving screen with more than one solvent. For example, a first cleaning of the moving screen can be realized with sprays of aqueous alcohol in a first cleaning section when the moving screen cross a first cleaning section and a second cleaning can be realized with water for example when said moving screen crosses a second cleaning section. A cleaning section contains at least a rail of cleaning solvent sprayers, the rail being perpendicular to movement of the moving screen. The cleaning section also includes the means to supply the fresh cleaning solvent and means to recover, recycle and/or treat the cleaning solvent once its cleaning action has been realized.

Extraction Solvent

The preferred extraction solvent of the process according to the present invention is aqueous alcohol. The alcohol is preferably ethanol. The amount and composition of the aqueous ethanol used to extract carbohydrates from the defatted and wetted flakes depends on final product requirements and to the exact type of defatted vegetable material that is processed. In general, it has been found that when the ethanol contains more water, it is more effective in extracting carbohydrates. Accordingly, less extraction solvent is required and a more concentrated miscella will result. However, a less concentrated aqueous ethanol is also a better solvent for proteins and its use therefore causes the protein content of the final protein concentrate to be reduced. It has been found that aqueous ethanol of 30:70 ratio is a good solvent for the extraction of soluble carbohydrates of soybean for example. This ratio is a good starting point for the extraction of the soluble carbohydrates of other vegetable materials as well form which minimal adjustments can be realized easily if needed. Extraction solvent temperature is usually close but just below to the atmospheric boiling point of the aqueous alcohol used (or its eutectic). For example, for aqueous ethanol of a 30:70 ratio, the preferred temperature is about 70° C. In order to reduce somehow the energy demand for the evaporation of the aqueous alcohol used as extraction solvent, this one can be displaced by concentrated alcohol. Indeed, the aqueous alcohol contains a substantial fraction of water which require substantial energy for its evaporation. Of course, this displacement takes place at the end of the extraction process per se.

Aqueous Alcohol Evaporation

Finally, the solvent wet vegetable protein concentrate leaving the extractor can be desolventized in standard desolventizers supplying indirect heat or direct heat by means of steam, or both. During desolventization, time, moisture and temperature are critical parameters with respect to protein denaturation, the acceptable magnitude of which is governed by the final product specification. The desolventized product may be dried and cooled before being further converted into the final product for sale. This conditioning may comprise, grinding, classification, blending and packaging.

Since the latent heat of evaporation of the aqueous alcohol solvent used in the process according to the present invention is high (40 kJ/mol or 2.2 kJ per gram for water and 38 kJ/mol or 0.83 kJ per gram for ethanol as opposed to 29 kJ/mol or only 0.34 kJ per g for hexane), it is advantageous to minimize the amount of solvent that has to be evaporated. Therefore, optionally, since at this stage of the process, the integrity of the vegetable protein concentrate is no longer critical, a desolventizing press can be used to squeeze out as much as possible of the solvent contained in the solvent-wet protein concentrate, provided this press has been constructed in an explosion proof manner. The solvent mixture leaving the press, having almost the same composition as the extraction solvent can profitably be returned to said extractor.

Treatment of the Miscella Containing the Soluble Carbohydrates

The extraction of the soluble carbohydrates leads also to a miscella comprising ethanol, water and carbohydrates; it may also contain limited amounts of proteins and other oleaginous seed components that are slightly soluble in aqueous ethanol. Extracted carbohydrates are recovered as molasses by evaporating the ethanolic solvent which is recycled in the extraction process. The carbohydrates may be isolated as a dry solid by evaporating the water contained in the molasses. Alternatively, molasses can be fermented into ethanol. In fact, the precise fate of the molasse often depends of local circumstances in order to optimize their utilization(s) by finding the most suitable nearby outlet(s). In some cases, the only possibility is to burn the molasse to produce energy.

Moving Screen Extractors

Good results have been obtained with “LM” type, moving screen solvent extractor (Desmet Ballestra, Belgium). However, the process according to the present invention is not limited to this precise model or supplier. All percolation extractors using a moving screen and equipped with adequate cleaning section able to clean the belt during the return journey of said moving screen are potentially suitable for the process according to the present invention. On a pilot solvent extractor of the type LM, no plugging of the moving screen has been observed for an extended period of time. For example, soluble carbohydrates of defatted soybean (white-flakes) have been extracted with aqueous ethanol (30:70 ethanol) in this pilot solvent extractor for 2 months, the moving screen being washed with a moderate output of 750 liters of water per minutes and per M². This amount of water was equally sprayed on both sides of the moving screen during its return journey. The total surface cleaned was is fact 0.1 m2 or 2.5% of the total surface of the moving screen (taking into account both sides of the moving belt-screen). Cleaning solvent was regular tap water having a temperature of approximatively 20° C. Pressure of the water was 0.3 bars. In those conditions absolutely, no plugging and/or no contamination or deposit of any sort was visible after the cleaning. The moving belt-screen retained its nominal solvent permeability during this extended period. The experiment was stopped for time constrain after two months, but the experiment could have been pursued a much longer time.

After cleaning of the moving screen with water, the cleaning solvent was switched to the aqueous alcohol used for the extraction of the defatted vegetable material. Again, the cleaning efficiency has been excellent for two months. After this period the experiment had to be stopped due to time constrain. But since after two months no contamination whatsoever on the moving screen was visible, it is highly probable that the moving screen would have stay clean for a much more extended period of time.

In another experiment the bottom sprayers have been switched off so that only the top sprayers were spraying cleaning solvent on the top side of the moving screen. Good cleaning of the moving screen has been observed but the experiment was only realized for 8 hours. The advantage of having only the top side of the moving screen cleaned is an overall reduction of cleaning solvent output with similar cleaning performance.

In another experiment the output and the pressure of the cleaning solvent was pulsated at the frequency of one hertz. Again, good cleaning results have been observed for a period of 8 hours. The advantage of the pulsation of the cleaning solvent output and pressure is an overall reduction of cleaning solvent output with similar cleaning performance.

The extractor has been used without any spray of solvent to determine the intensity of the plugging of the moving screen without the continuous cleaning of the moving screen according to the present invention. The plugging appears significantly and rapidly. The onset of the percolation decrease starts already after 5 hours of operation. Even if the decrease of the percolation was moderate just after this initial onset, it progressively decreases to unacceptable level. Percolation was stopped after 24 hours of continuous operation. It must be outlined that at this stage of contamination and plugging, the mild cleaning conditions, as disclosed in the present invention, are not aggressive enough to remove the deposits plugging the moving screen. Indeed, the residue had the time to coagulate and dry during the successive return journeys of the moving screen. Therefore, they form a compact mass adhering strongly to the moving screen and its cleaning was only possible with aggressive and repeated high-pressure water sprayed at 150 bars. Such aggressive cleaning can only be done manually and when the extractor is on hold and emptied of any material and solvent for obvious safety reasons. This reference test has been performed at the end of the other two experimentations because of the fear of damaging the equipment with the extend of the plugging of the moving screen and the necessary intense and aggressive cleaning that was expected. To this respect it is important to understand that when such intense and aggressive cleaning become necessary, it is mandatory to fully stop the extractor, drain all solvent and remove any vegetable material that may be loaded in the extractor. The high-pressure cleaning will also induce projection of the built-up and other deposits that have accumulated on other parts of the extractor and consequently those projections have to be cleaned as well. Furthermore, there is always the risk of causing damage to the extractor during such intensive and aggressive cleaning. In sharp opposition, the process according the present invention use a very mild but continuous cleaning. The cleaning solvent sprays on the moving screen are done at a much lower pressure but are more efficient to continuously dislodge any fresh build-up or any other deposits before they start to adhere to said moving screen. Those mild cleaning solvent sprays does not induce projection on any other parts of the extractor. Furthermore, since the continuous cleaning as described in the present invention is not aggressive there is no risk of damages to the equipment. With such moving screen extractor, the size of the extractor should be bigger because only the top deck of the extractor is used for the extraction process. However, since the moving screen is constantly maintained clean and thus keep its nominal solvent percolation rate, the maximal thickness of material can be permanently loaded on said moving screen. Furthermore, the fact that not manual cleaning is necessary for extended period of time largely compensate the fact that the use of a moving screen extractor instead of a fixed screen extractor may necessitate a larger capital investment.

Without willing to be bound by the theory, it is also believed that the efficiency of the cleaning of the moving screen according to the present invention is due to the fact that said cleaning can be concentrated on a relatively small zone of the moving screen, typically, a zone of about 0.25 to 2 M², the exact surface depending on the global size of the extractor. The cleaning on such a limited zone is therefore localized and concentrated on a small surface hence efficient. Once cleaned, the moving screen stays clean during the remaining part of its return journey and thus maintain its full permeability allowing the maximum percolation rate once said moving screen reaches again the extraction zone of the extractor. In sharp contrast, the cleaning of a fixed screen involves the cleaning of a very large surface, of for example 150 m² or more in the case of large extractor. Hence, the cleaning cannot be intense enough to remove systematically any build-up material sticking on the fixed screen leading thus to a much more rapid clogging of said fixed screen. Furthermore, the cleaning solvent will mix with the miscella offering less flexibility for the choice of the nature of the cleaning solvent, its output, its pressure and its temperature.

1. A process, for the production of vegetable protein concentrate from a vegetable defatted material including the following steps:
a) removing at least a fraction of the soluble carbohydrates of said vegetable defatted material by solvent extraction, the solvent being aqueous alcohol, to obtain solvent wet protein rich vegetable material and a miscella,
b) removing the aqueous alcohol solvent from the solvent wet protein rich vegetable material to obtain a vegetable protein concentrate,
wherein the solvent extraction takes place in a moving screen extractor equipped with a cleaning means of the moving screen during its return journey said cleaning means including contacting cleaning solvent with said moving screen and insuring a complete cleaning of both sides of said moving screen during the return journey of said moving screen and thus avoiding the progressive plugging of said moving screen and therefore insuring continuous operation for an extended period of time.

2. The process according to claim 1 wherein said extended period of continuous operation of the moving screen extractor is superior to two months.

3. The process according to claim 1 wherein said extended period of continuous operation of the moving screen extractor is superior to three months.

4. The process according to claim 1 wherein said extended period of continuous operation of the moving screen extractor is superior to four months.

5. The process according to claim 1 wherein said extended period of continuous operation of the moving screen extractor is superior to five months.

6. The process according to claim 1 wherein said extended period of continuous operation of the moving screen extractor is superior to six months.

7. The process according to claim 1 wherein said cleaning means include the use sprayers projecting cleaning solvent towards said moving screen.

8. The process according to claim 1 wherein said cleaning means include scrapers and brushes to mechanically clean the moving screen.

9. The process according to claim 1 wherein said cleaning means is installed at the very beginning of return journey of said moving screen.

10. The process according to claim 1 wherein the cleaning of said moving screen is repeated two or more times by installing two or more cleaning elements in series on the return journey of said moving screen.

11. The process according to claim 1 wherein said cleaning solvent has the same composition than the aqueous alcohol used as extraction solvent for the soluble carbohydrates.

12. The process according to claim 1 wherein said cleaning solvent has the same composition than the aqueous alcohol used as extraction solvent for the soluble carbohydrates and is recycled in the last extraction zone of said solvent extractor.

13. The process according to claim 1 wherein said cleaning solvent is water.

14. The process according to claim 7 wherein, the pressure of the solvent spray is pulsating alternating for example very low pressure comprised between 0.1 to 5.0 bars and higher pressure comprised between 5.0 and 100.0 bars.

15. The process according to claim 1 wherein the vegetable defatted material is obtained from the deoiling of oleaginous vegetable material such as soybeans, sunflower or rapeseed.

16. The process according to claim 1 wherein the vegetable defatted material is selected amongst legumes such as lentils, beans or peas said legume having optionally been pretreated with treatment such as dehulling, flaking, cooking.

17. The process according to claim 1 further characterized by using a cleaning solvent bath and immerging at least a part of the moving screen in the cleaning solvent bath said immersion occurring during the return journey of said moving screen.

18. The process according to claim 1 wherein the moving screen is cleaned and maintain its nominal solvent percolation capacity by sprays of cleaning solvent projected on said moving screen during its return journey and wherein the cleaning solvent does not mix with said miscella.

19. The process according to claim 1 wherein the moving screen is cleaned and maintain its nominal solvent percolation capacity by upwards and downwards sprays of cleaning solvent projected on said moving screen during its return journey.

20. The process according to claim 1 wherein the moving screen is cleaned and maintain its nominal solvent percolation capacity by downwards sprays of cleaning solvent projected on said moving screen during its return journey.

21. The process according to claim 1 wherein, said cleaning means includes at least one rail of cleaning solvent sprayers, said rail being essentially perpendicular to the movement of said moving screen.

22. The process according to claim 1 wherein said cleaning means includes a plurality of cleaning solvent sprayers, said sprayers projecting cleaning solvent at a pressure ranging from 0.1 to 3.0 bars.

23. The process according to claim 1 wherein said cleaning means includes a plurality of cleaning solvent sprayers, said sprayers projecting cleaning solvent at an output of 150 to 1500 liters per minutes and per M².

24. The process according to claim 1 wherein the defatted vegetable material is introduced on the moving screen of the moving screen extractor in a way to cover only a fraction of the width of said moving screen, said defatted vegetable material being subsequently wetted by the extraction solvent and allowed to swell freely at least in the transversal direction and said defatted vegetable material introduced on the moving screen of the moving screen extractor being dry or already wetted by a solvent such as concentrated alcohol, aqueous alcohol or hexane.
